(19) United States
(12) Patent Application Publication
(10) Pub. No.: US 2014/0249533 A1
(43) Pub. Date: Sep. 4, 2014

(54) Rongeur

(71) Applicant: Phillip J. Agoston, Byrdstown, TN (US)

(72) Inventor: Phillip J. Agoston, Byrdstown, TN (US)

(21) Appl. No.: 14/193,650

(22) Filed: Feb. 28, 2014

Related U.S. Application Data
(60) Provisional application No. 61/835,230, filed on Jun. 14, 2013.
(60) Provisional application No. 61/771,522, filed on Mar. 1, 2013.

(51) Int. Cl. A61B 17/16

(52) U.S. Cl. CPC A61B 17/1606
USPC 606/83

(57) ABSTRACT

A rongeur is disclosed having a stationary shank and a crossbar slideable relative to the shank with a tongue in crossbar cooperating with a channel in the shank so as to interconnect the shank to the crossbar. At least one gap section is formed in both the shank and the crossbar to gain access to the interior of the rongeur to permit cleaning and lubrication without disassembling and reassembling the rongeur. Multiple gap sections can be placed in the rongeur longitudinally along the shaft and crossbar if desired.

### CROSS-REFERENCE TO RELATED APPLICATIONS

The present Application claims the benefit of U.S. Provisional Application No. 61/835,230, filed Jun. 14, 2013, and U.S. Provisional Application No. 61/771,522, filed Mar. 1, 2013, the contents of which are incorporated herein by reference.

### FEDERALLY SPONSORED RESEARCH OR DEVELOPMENT

N/A

### TECHNICAL FIELD

The present invention relates to a rongeur, and more particularly to a rongeur with access to its interior for cleaning and lubricating.

### BACKGROUND

A rongeur is a hand held surgical instrument used for removing small amounts of bone, cartilage, or other biomass and tissues from sites within a body. A typical rongeur includes a fixed shank with an anvil or footplate at its distal end and a handle at its proximal end. A cross bar slidably engages the shank and reciprocates thereon by a pivotable second handle. Cutting edges on the distal end of the crossbar engage and contact against the footplate to cut away a small portion of material and hold it between the footplate and cutting edge with each reciprocation of the crossbar.

As with most medical instruments, very small tolerances between the mating parts are mandated. Such tolerances often make cleaning extremely difficult, particularly the interior of such instruments. Blood and other bodily matter trapped between the shank and crossbar can be very difficult to remove. Failure to remove such matter can lead to incomplete sterilization. Accordingly, it is desirable to allow access to these parts during cleaning and sterilization.

Another problem with rongeurs is the inability to properly lubricate all of the internal contacting surfaces without completely disassembling the instrument. Lack of proper lubrication makes the instrument difficult, if not impossible, to use and shortens the life of the instrument. Accordingly, it is also desirable to gain access to these parts after sterilization for proper lubrication.

In sum, to properly clean and lubricate a rongeur, it must be assembled and then reassembled. This can be time consuming. Accordingly there is a desire to both clean and lubricate rongeurs without the need of disassembling and reassembling them.

It is believed that the rongeur of the present invention alleviates many of these issues. The rongeur includes a shank and crossbar and incorporates gaps without them so as to gain easy access to the interior of the rongeur without disassembly. Significantly, existing rongeurs can be modified with and by the teachings of the present invention so as to make their interiors more accessible for cleaning and lubricating.

### BRIEF DESCRIPTION OF THE DRAWINGS

To understand the present invention, it will now be described by way of example, with reference to the accompanying drawings in which:

FIG. 1 is a side elevation of an embodiment of an assembled rongeur of the present invention;

FIG. 2 is a top plan view of the distal end of the rongeur of FIG. 1;

FIG. 3 is a bottom plan view of the crossbar of the rongeur of FIG. 1 with the shank removed therefrom;

FIG. 4 is a side elevation view of the crossbar of the rongeur of FIG. 3;

FIG. 5 is a top plan view of the shank of the rongeur of FIG. 1 with the crossbar removed therefrom;

FIG. 6 is a side elevation view of the shank of the rongeur of FIG. 5.

FIG. 7 is an enlarged side elevation view of the shank of the rongeur of FIG. 10;

FIG. 8 is an enlarged side elevation view of the crossbar of the rongeur of FIG. 10;

FIG. 9 is an enlarged top view of the crossbar of the rongeur of FIG. 10;

FIG. 10 is a side elevation of an embodiment of an assembled rongeur of the present invention; and

FIG. 11 is a top plan view of the crossbar of the rongeur of FIG. 10.

### DETAILED DESCRIPTION

The present invention is directed to an improved medical rongeur that is more readily flushed, cleaned, sterilized, dried, and lubricated, than those commonly used. While the present invention is directed generally to rongeurs, it applies to many specific types of rongeurs such as Kerrison, Lempert, Schlesinger, Cushing, Stille-Horsley or Sella Punch Rongeurs. The present invention can be made or modified with rongeurs having a variety of sizes and shapes as are well known in the art. In particular, the present invention is useful with rongeurs having a variety of different cutting surfaces, sizes and shapes as well as different sizes, configurations and shapes for the handles and fixed shanks. The present invention also applies to other types of medical instruments that have inaccessible surfaces that are difficult, if not impossible, to properly flush, clean, sterilize, dry or lubricate.

As shown in FIGS. 1 and 10, the rongeur 10 of the present invention includes a standard handle 11 with a front grip 12 and rear grip 13 and a biasing mechanism 14 disposed therebetween. The front grip is interconnected to the rear grip 13 by a screw 15 or similar fastener. A fixed shank 20 is integral with the rear grip 13 and a crossbar 40 is interconnected via a screw 16 to the front grip 12. At the distal end of the crossbar 40, there is a cutting plane 41 and the end of the distal end of the shank 20 there is a footplate 21. Movement of the front grip 12 relative to the fixed rear grip 13 by a squeezing motion causes the crossbar 40 to move relative to the shank 20. Specifically, when the grip 12 is moved in the direction marked as direction A towards the grip 13, the crossbar 40 moves in the direction marked A relative to the shank 20. Similarly, movement of the front grip 12 in the direction B results in movement of the crossbar in the direction B relative to the shank.

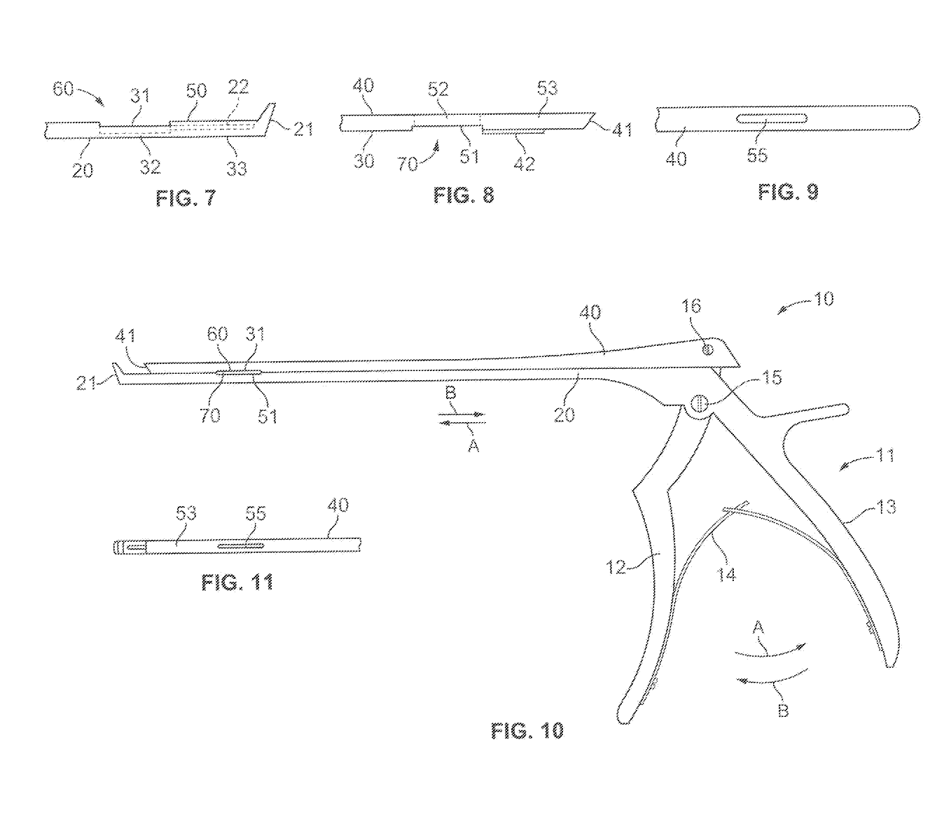

With respect to the embodiment shown in FIGS. 7-11, the crossbar 40 is slidably connected to the shank 20 by means of a tongue 42 projecting downwardly from the head section 53 of the crossbar into a receiving channel 22 in the head section 33 of the shank. Both the tongue 42 and the receiving channel 22 can be keyed for ensuring they stay interconnected. The shank 20 further has a sliding surface 50 abutting the sliding surface 30 of the crossbar 40. Adjacent the head section 53 of the crossbar 40 is a gap section 70 comprised of a cut-out 51 in the crossbar resulting in a relatively thinner portion 52 formed in the crossbar 40. Similarly, adjacent the head section 33 of the shank 20, there is a gap section 60 comprised of a notch or cut-out 31 in the shank resulting in a relatively thinner portion 32 formed in the shank 20. A hob or channel 55 is also formed in the top surface of the crossbar 40. The hole/channel 55 may communicate with the gap sections 60,70.

More particularly, to form the gap section 70 in the crossbar 40, each side of the crossbar is cut-out towards the top surface of the crossbar so as to leave a relative thin, yet strong, longitudinal section 52 connecting the proximate end of the crossbar to its distal end, that being the section supporting the cutting plane 41. To form the gap section 60 in the shank 20, each side of the shank is cut-out towards the bottom surface of the shank so as to leave a relatively thin, yet strong, longitudinal section 32 connecting the proximate end of the shank to its distal end, that being the footplate 21.

The two gap sections 60,70 basically align with one another when the rongeur 10 is at rest, as shown in FIGS. 1 and 10. By being aligned, the two gaps sections 60,70 form a single opening into the center of the rongeur in communication with the hole/channel 55. The outermost surface of the longitudinal sections 52,32 can also be shaved or cut so that a space or opening 80 is formed between the two gap section 60,70.

The set of gap sections 60,70 permit access to the inside of the rongeur while it is still assembled (FIGS. 1 and 10) facilitating the cleaning and lubrication thereof. If the crossbar is moved relative to the shank, while assembled, the interior can be further cleaned or lubricated.

While only one set of the gap sections 60,70 is shown, it is recognized that multiple sets of gap sections 60,70 can be placed in the rongeur 10 longitudinally along the shaft 20 and crossbar 40 if desired. Multiple channels 55 may also cooperate and communicate the multiple gap sections 60,70.

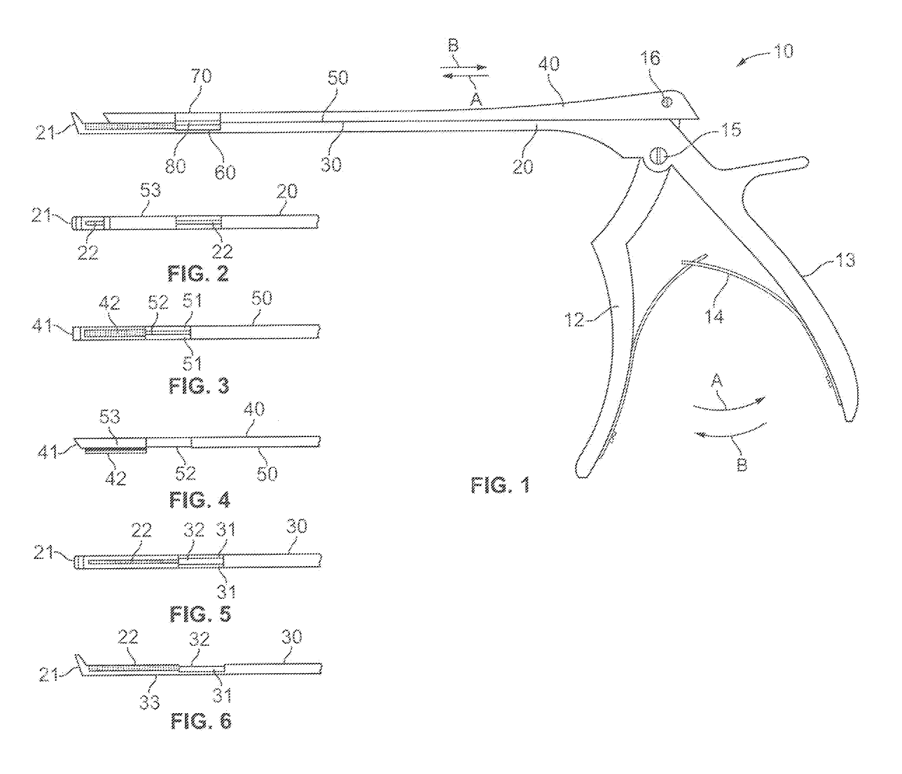

In another embodiment shown in FIGS. 1-6, the crossbar 40 is slideably connected to the shank 20 by means of a tongue 42 projecting downwardly from the head section 53 of the crossbar into a receiving channel 22 in the head section 33 of the shank. Both the tongue 42 and the receiving channel 22 can be keyed for ensuring they stay interconnected. The shank 20 further has a sliding surface 50 abutting the sliding surface 30 of the crossbar 40. Adjacent the head section 53 of the crossbar 40 is a gap section 70 comprised of two notches or cut-outs 51 in the crossbar resulting in a relatively thinner portion 52 formed in the crossbar 40. Similarly, adjacent the head section 33 of the shank 20, there is a gap section 60 comprised of two notches or cut-outs 31 in the shank resulting in a relatively thinner portion 32 formed in the shank 20.

More particularly, to form the gap section 70 in the crossbar 40, each side of the crossbar is cut-out or shaved inwardly towards the centerline of the crossbar so as to leave a relative thin, yet strong, longitudinal section 52 connecting the proximate end of the crossbar to its distal end, that being the section supporting the cutting plane 41. To form the gap section 60 in the shank 20, each side of the shank is cut-out or shaved inwardly towards the centerline of the shank so as to leave a relatively thin, yet strong, longitudinal section 32 connecting the proximate end of the shank to its distal end, that being the footplate 21.

The two gap sections 60,70 basically align with one another when the rongeur 10 is at rest, as shown in FIG. 1. The outermost surface of the longitudinal section 52 can also be shaved or cut so that a space or opening 80 is formed between the two gap sections 60,70.

The set of gap sections 60,70 permit access to the inside of the rongeur while it is still assembled (FIG. 1) facilitating the cleaning and lubrication thereof. If the crossbar is moved relative to the shank, while assembled, the interior can be further cleaned or lubricated.

While only one set of the gap sections 60,70 is shown, it is recognized that multiple sets of gap sections 60,70 can be placed in the rongeur 10 longitudinally along the shaft 20 and crossbar 40 if desired. Multiple channels 55 may also cooperate and communicate the multiple gap sections 60,70.

It should also be noted that the present invention can be made into the rongeur when it is being constructed, or an existing rongeur can be modified so as to have one or more gap sections along she shaft and crossbar.

The terms “first,” “second,” “upper,” “lower,” “top,” “bottom,” “above,” “below,” etc. are used for illustrative purposes to associate relative positioning of elements to other elements only and are not intended to limit the embodiments in any way. The term “plurality” as used herein is intended to indicate any number greater than one, either disjunctively or conjunctively as necessary, up to an infinite number. The terms “joined” “attached,” and “connected” as used herein are intended to put or bring two elements together so as to form a unit, and any number of elements, devices, fasteners, etc. may be provided between the joined or connected elements unless otherwise specified by the use of the term “directly” and/or supported by the drawings.

While the specific embodiments have been illustrated and described, numerous modifications come to mind without significantly departing from the spirit of the invention, and the scope of protection is only limited by the scope of the accompanying Claims.

What is claimed is:

1. A rongeur comprising:
a stationary shank;
a crossbar slideable relative to the shank, the crossbar having a rest position;
a tongue in one of the shank and crossbar cooperating with a channel in the other of the shank and crossbar so as to interconnect the shank to the crossbar;
a gap section in the shank;
a gap section in the crossbar disposed adjacent the gap section in the shank when the rongeur is in the rest position.

2. The rongeur of claim 1 wherein the gap section of the shank is formed by at least one cut-out section in the shank.

3. The rongeur of claim 2 wherein the gap section of the crossbar is formed by at least one cut-out section in the crossbar.

4. The rongeur of claim 1 wherein the gap section of the shank is formed by two cut-out sections in the shank resulting in a thinner portion, and the gap section of the crossbar is formed by two cut-out sections in the crossbar resulting in a thinner portion.

5. The rongeur of claim 4 wherein the shank has a plurality of gap sections, and the crossbar has a plurality of gap sections disposed adjacent the gap sections in the shank.

6. The rongeur of claim 1 wherein the crossbar has a channel in its top surface, the channel cooperating with the gap sections of the shank and crossbar.

7. The rongeur of claim 5 wherein the crossbar has a plurality of channels in its top surface, each of the plurality of channels cooperating with gap sections of the shank and crossbar.
